(19) United States
(12) Patent Application Publication
(10) Pub. No.: US 2014/0005575 A1
(43) Pub. Date: Jan. 2, 2014

(54) ACTIVITY METER

(71) Applicant: OMRON HEALTHCARE CO., LTD., Muko-shi (JP)

(72) Inventors: Hiroshi OGAWA, Nara-shi (JP); Yuji ASADA, Kyoto-shi (JP); Naoki TAKEISHI, Toyonaka-shi (JP); Yoshitake OSHIMA, Kyoto-shi (JP)

(73) Assignee: OMRON HEALTHCARE CO., LTD., Muko-shi (JP)

(21) Appl. No.: 13/959,399

(22) Filed: Aug. 5, 2013

Related U.S. Application Data
(63) Continuation of application No. PCT/JP2011/079347, filed on Dec. 19, 2011.

(30) Foreign Application Priority Data
Mar. 14, 2011 (JP) 2011-055468

(51) Int. Cl. A61B 5/11; A61B 5/00

(52) U.S. Cl. CPC A61B 5/1118; A61B 5/4866; A61B 5/7278; A61B 5/4872
USPC 600/595

(57) ABSTRACT

An activity meter has an activity amount acquisition unit that acquires an amount of physical activity of a user. The activity meter acquires a first index relating to a difference between target calories burned for a unit period and computed calories burned that are computed from the amount of activity acquired by the activity amount acquisition unit in the unit period, and acquires a second index relating to a difference between a target value representing a body composition and a measurement value representing a body composition that is measured for the user. Information on an evaluation of the amount of activity based on the first index and second index that have been acquired is output.

### TECHNICAL FIELD

The present invention relates to an activity meter that measures the amount of physical activity of a user, and more particularly to an activity meter that outputs information relating to the amount of physical activity.

### BACKGROUND ART

For activity meters, Patent Literature 1 (JP 2006-204446A) and Patent Literature 2 (JP 2001-258870A) show methods for measuring the exercise intensity of physical activity or the calories burned during physical activity utilizing an acceleration sensor. With the activity meter of Patent Literature 1 (JP 2006-204446A), a standard deviation Sw of acceleration in a fixed time period tw is computed from the output signal of the acceleration sensor, and an exercise intensity wi is computed from the standard deviation Sw using a conversion equation formulated in advance. Also, with the device of Patent Literature 2 (JP 2001-258870A), the impulse of momentum is calculated by vector synthesis from triaxial acceleration, and energy expenditure is calculated from the impulse in response to the type of exercise. The type of exercise is determined based on the ratio between the impulse calculated by vector synthesis and the impulse in the depth, horizontal and vertical directions.

In Patent Literature 3 (JP 2010-17525A), what age activity pattern the user's state of activity is equivalent to is computed by comparing the energy expenditure history with reference data.

Patent Literature 4 (JP 2008-250967A) shows a configuration in which dietary intake amount is determined from the computed amount of physical activity and the result thereof is displayed.

### CITATION LIST

### Patent Literature

Patent Literature 1: JP 2006-204446A
Patent Literature 2: JP 2001-258870A
Patent Literature 3: JP 2010-17525A
Patent Literature: JP 2008-250967A

### SUMMARY OF INVENTION

### Technical Problem

Although the activity meters of Patent Literature 1 (JP 2006-204446A) and Patent Literature 2 (JP 2001-258870A) output the user's activity amount as calories burned, it is unclear whether the amount of calories burned is high or low in comparison with a person of the same age or to a person of what age that amount of calories burned is equivalent. In order to compensate for this, in Patent Literature 3 (JP 2010-17525A), the age activity pattern to which the user's state of activity is equivalent is computed.

Meanwhile, although there is a demand for users to be able to personally evaluate the amount of activity (calories burned) and ask for advice to satisfy his/her health awareness due to a recent increased trend toward health awareness, the above-described conventional technology has not fulfilled such a demand.

Hence, an object of this invention is to provide an activity meter that outputs information for evaluating the amount of activity.

### Solution to Problem

The activity meter according to this invention includes an activity amount acquisition unit that acquires an amount of physical activity of a user, an acquisition unit that acquires a first index relating to a difference between target calories burned for a unit period and computed calories burned that are computed from the amount of activity acquired by the activity amount acquisition means in the unit period, an acquisition unit that acquires a second index relating to a difference between a target value representing a body composition and a measurement value representing a body composition that is measured for the user, and an output unit that outputs information on an evaluation of the activity amount based on the first index and the second index that have been acquired.

### Advantageous Effects of Invention

According to the present invention, it is possible to provide an activity meter that is capable of outputting information for evaluating the amount of activity.

### BRIEF DESCRIPTION OF DRAWINGS

FIGS. 1A and 1B are diagrams respectively illustrating the external appearance and a mode of wearing an activity meter according to an embodiment of the present invention.

FIG. 2 is a diagram showing the hardware configuration of a system that includes an activity meter according to an embodiment of the present invention.

FIG. 3 is a diagram showing the functional configuration of an activity meter according to an embodiment of the present invention.

FIG. 4 is a diagram showing exemplary memory content of a memory according to an embodiment of the present invention.

FIG. 5 is a processing flowchart according to an embodiment of the present invention.

FIG. 6 is a diagram showing an example of display according to an embodiment of the present invention.

FIG. 7 is a diagram showing another example of display according to an embodiment of the present invention.

FIG. 8 is a flowchart for computing an aging index according to an embodiment of the present invention.

FIG. 9 is diagram showing an example of a table of messages corresponding to aging indices according to an embodiment of the present invention.

### DESCRIPTION OF EMBODIMENTS

Hereinafter, embodiments of the present invention will be described in detail with reference to the drawings. Note that the same or corresponding portions in the following embodiments are given the same reference signs in the drawings, and description thereof will not be repeated.

First, terms used in the present embodiment will be described. In the present embodiment, “activity age” represents a standard (or average) age of persons who, in a predetermined period, burn the total amount of calories burned by the user when active for the same period. Here, for ease of description, the predetermined period is 1 day.

Also, “real age” indicates chronological age (age counted from the time of birth). Further, “body age” indicates biological age, more specifically it indicates the age of a body based on it's physical composition. It is assumed that the basal metabolic rate is used as a typical example of information for physical composition, but the present invention is not limited to thereto.

In the present embodiment, METs (Medical Evangelism Training & Strategies) is used as an index indicating physical activity intensity. A METs is a unit representing the intensity of physical activity in multiples of a resting state, with sitting down quietly being equivalent to 1 METs and normal walking being equivalent to 3 METs.

Also, “exercise (Ex)” is a unit representing the amount of physical activity, and is obtained by multiplying the intensity of physical activity (METs) by the implementation time period (time: hour) of physical activity.

In the present embodiment, a device that measures the number of steps a person takes is illustrated as the activity meter, but the activity meter is not limited to such a device. In other words, the activity meter can be any device having a function capable of measuring the activity amount resulting from physical activity including exercise and daily activities (e.g., vacuuming, carrying items, cooking, etc.). Although the activity meter can be shared among two or more persons, it is assumed here for ease of description that the activity meter is used by one person.

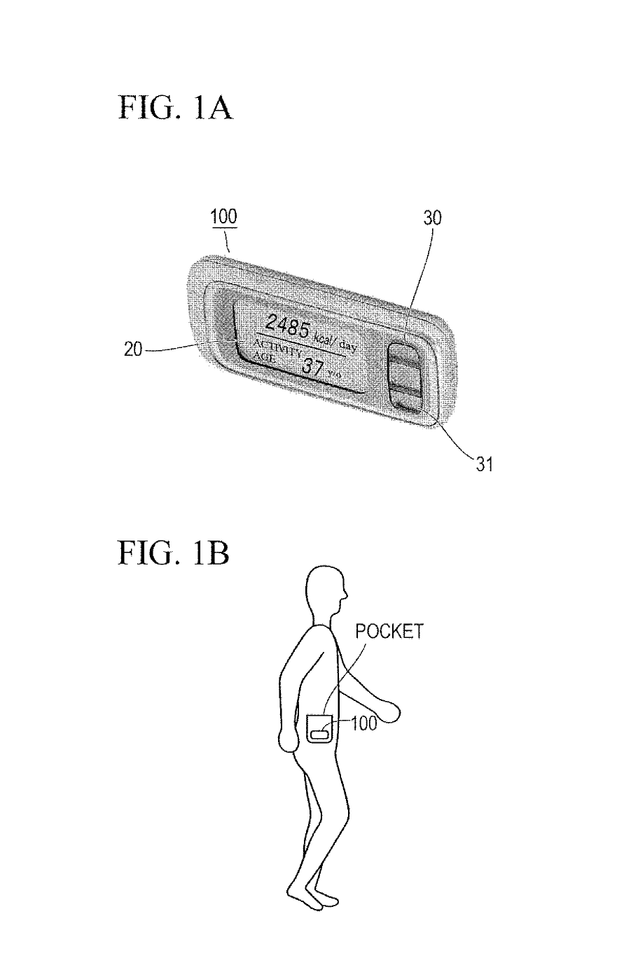

Referring to (A) of FIG. 1, a casing of an activity meter 110 is provided with a display 20 capable of displaying various items of information such as the counted number of steps, activity intensity and activity age, and an operation unit 30 having various buttons with which a user can perform operations. The operation unit 30 includes a button 31 that the user operates in order to request output of the activity age.

The user performs physical activity including exercise and daily activities while carrying the activity meter 100 in a pocket or the like in clothes, as shown in (B) of FIG. 1.

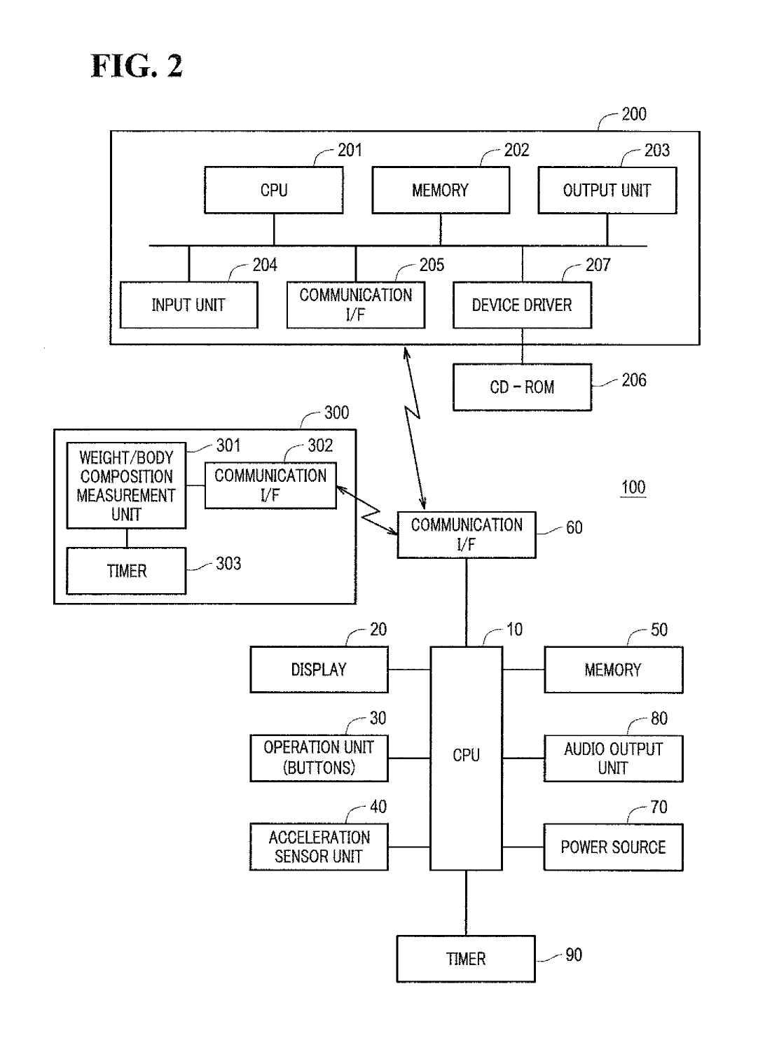

Referring to FIG. 2, a hardware configuration will now be described with reference to a system including the activity meter 100. The system includes the activity meter 100 and external devices 200 and 300 that communicate with each other.

The activity meter 100 includes, as hardware, for example, a CPU (Central Processing Unit) 10 for performing overall control, the display 20, the operation unit 30, an acceleration sensor unit 40 including an acceleration sensor and an MPU (Micro-Processing Unit), a memory 50 for storing programs that are executed by the CPU 10, data and the like, a communication I/F (abbreviation of “interface”) 60 for wireless or wired communication with an external device, a power source 70 such as a battery, an audio output unit 80 for outputting audio, and a timer 90 that clocks time and outputs time data.

The activity meter 100 performs wireless or wired communication with external devices 200 and 300 via the communication I/F 60. The device 200 is equivalent to a mobile terminal (PDA (Personal Digital Assistant), mobile phone, etc.) or a stand-alone computer, for example, and the device 300 has a function of measuring the user's weight and body composition. Here, body composition indicates physical composition such as muscle mass, bone mass, fat mass, and the like of body.

The device 200 includes, as hardware, for example, a CPU 201, a memory 202, an output unit 203, an input unit 204, a communication I/F 205, and a device driver 207 for accessing data in a CD-ROM (Compact Disk Read Only Memory) 206. The device driver 207 has the CD-ROM 206 removably loaded therein, and reads out data (including programs) from the loaded CD-ROM 206 or writes data to the loaded CD-ROM 206.

The device 300 includes a weight/body composition measurement unit 301 that measures the user's height, weight, body composition (body fat and the like), a communication I/F 302 for transmitting measured information to outside the device, and a timer 303. Measured weight and body composition information is transmitted to the activity meter 100 via the communication I/Fs 302 and 60, as weight data and body composition data to which time data indicating the measurement time clocked by the timer 303 is respectively added. The timer 303 and the timer 90 are adjusted so as to perform synchronized clocking operations. The weight/body composition measurement unit 301 has a scale function and measures the weight of the user. Also, the weight/body composition measurement unit 301 has a function for computing values of body composition based on bioelectrical impedance measured from the user.

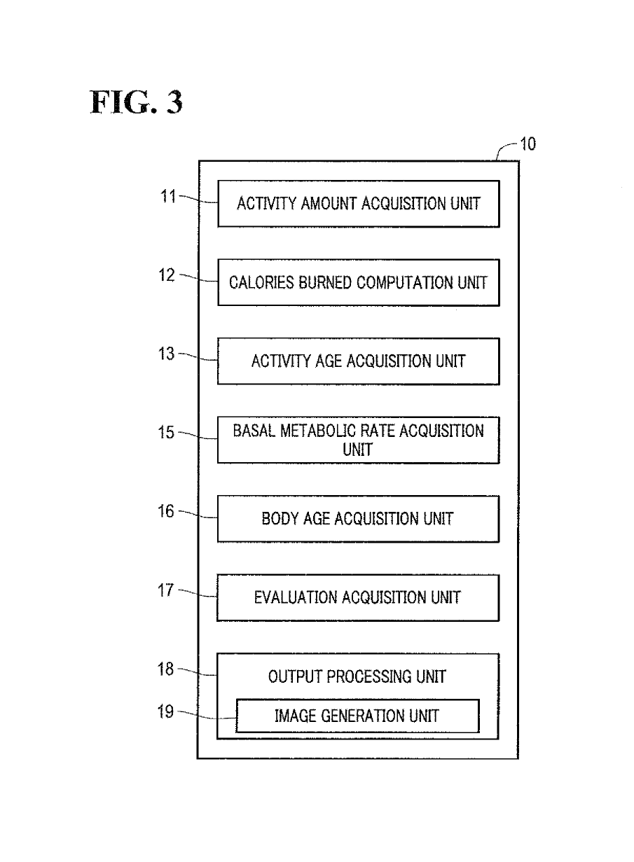

The configuration of functions that operate under the control of the CPU 10 is shown in FIG. 3. The functions include an activity amount acquisition unit 11 for acquiring the amount of physical activity of the user, a calories burned computation unit 12 for computing the total calories burned in the predetermined period based on the acquired activity amount, an activity age acquisition unit 13 for acquiring the activity age from the computed calories burned in the predetermined period, a basal metabolic rate acquisition unit 15 for acquiring the basal metabolic rate, a body age acquisition unit 16 for acquiring the body age, an evaluation acquisition unit 17 for acquiring information for evaluating the amount of activity, and an output processing unit 18 for outputting various items of information to outside the device. The information output from the output processing unit 18 includes an image, and the output processing unit 18 includes an image generation unit 19 that generates the image. These units are equivalent to a program or a combination of a program and a circuit module.

### Computation of Calories Burned Based on Activity Amount

The activity amount acquisition unit 11 receives input of activity intensity from the acceleration sensor unit 40 and input of time data from the timer 90. Activity intensity data Mi (discussed later) obtained by associating the activity intensity from the acceleration sensor unit 40 and the time data from the timer 90 is acquired, and the acquired activity intensity data Mi is stored in the memory 50. The time data associated with the activity intensity indicates the implementation date and time of the exercise for which the activity intensity was measured.

The acceleration sensor unit 40 measures the number of steps similarly to measurement of the number of steps by a generic pedometer. The acceleration sensor detects acceleration applied to the activity meter 100. The detected acceleration is derived as a voltage signal. The MPU processes the output signal from the acceleration sensor. For example, the MPU performs processing so as to count each time an acceleration of greater than or equal to a threshold is detected as one step, based on the signal output sequentially from the acceleration sensor.

The measurement operation performed by the MPU of the acceleration sensor unit 40 involves computing the activity intensity (unit: METs) per unit period, using acceleration data measured based on the acceleration signal input from the acceleration sensor, with predetermined time intervals (e.g., 20-second intervals, etc.) defined in advance as the unit period. As a specific computation method, for example, the activity intensity can be computed using a well-known technique, such as the technique disclosed by the applicant in JP 2009-28312A.

Activity intensity is an index representing the intensity of physical activity that depends on walking pitch (number of steps per unit period) and the height of the user that is input in advance. For example, a resting state is equivalent to 1 METs, walking normally (4 km/h) is equivalent to 3 METs, vacuuming is equivalent to 3.5 METs, and jogging is equivalent to 7 METs (from Exercise and Physical Activity Guide for Health Promotion “Exercise Guide 2006” (Ministry of Health, Labour and Welfare)).

The activity intensity may be computed by a method using the heart rate detected from the user and a predetermined arithmetic equation, instead of being computed by the abovementioned method based on body motion detected in accordance with the acceleration signal.

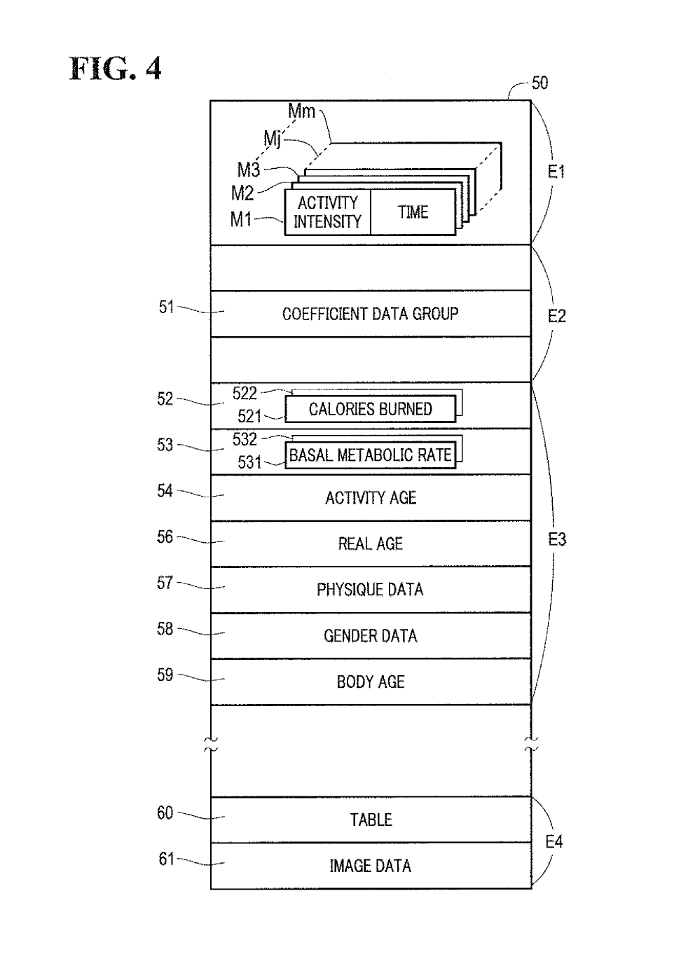

The storage content of the memory 50 is illustrated in FIG. 4. Referring to FIG. 4, the memory 50 includes an area E1 where the activity intensity data Mi (i=1, 2, 3, . . . n) is stored, an area E2 where a coefficient data group 51 that consists of different types of coefficients that are used in the conversion equation for converting total calories burned into an activity age is stored, an area E3, and an area E4 for storing a table 60 and image data 61. The coefficients in the coefficient data group 51 are assumed to have been computed in advance through testing and stored.

The activity intensity data Mi includes measured activity intensity and a measurement time period indicating the time period for which the activity was implemented. The values of the coefficients in the coefficient data group 51 may be variably set by a user operation via the operation unit 30.

The area E3 has stored therein computed calories burned 52 for the user, a computed basal metabolic rate 53 and an acquired activity age 54, and further has stored therein a real age 56 of the user, physique data 57 including the user's weight, height and the like, gender data 58 indicating the gender of the user, and a body age 59 of the user. Here, the real age 56, the physique data 57 and the gender data 58 represent body information relating to the user's body.

The image data 61 of the area E4 includes data to be used for generating an image to be displayed on the display 20, and data to be generated and displayed.

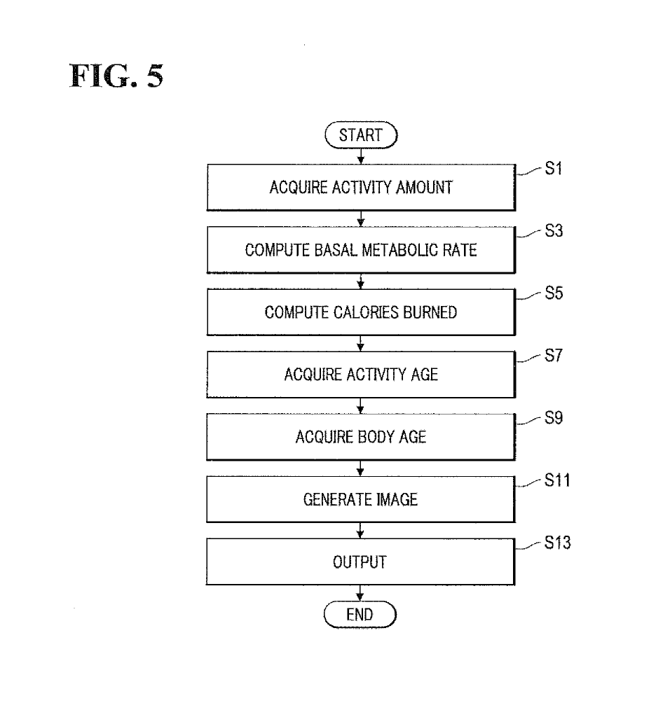

A processing flowchart according to the present embodiment is shown in FIG. 5. Processing according to this processing flowchart is realized by the CPU 10 reading out a predetermined program from the memory 50 and executing the instructions of the read program. Computation of the user's activity age and body age and output of information will be described according to the flowchart of FIG. 5. Note that it is assumed that a sufficient number of sets of activity intensity data Mi are stored in the area E1 of the memory 50.

When the user operates the button 31 of the operation unit 30, the CPU 10 receives the operation. Specifically, based on the operation signal output from the operation unit 30 as a result of the button 31 being operated, the CPU 10 starts the processing of FIG. 5. When the processing has been started, the activity amount acquisition unit 11 acquires the activity amount for the predetermined period, based on the activity intensity data Mi read out from the area E1 of the memory 50 (step S1). Subsequently, the basal metabolic rate acquisition unit 15 computes a basal metabolic rate of the user for a predetermined period (step S3). The procedure for computing a basal metabolic rate will be described in detail later. The calories burned computation unit 12 then computes the total calories burned for the predetermined period in accordance with the above-described computation equation, based on the acquired activity amount (step S5).

The activity age acquisition unit 13 computes the activity age, in accordance with a predetermined conversion equation using the computed total calories burned and basal metabolic rate and coefficients of the coefficient data group 51 (step S7). The procedure for computing the activity age will be described in detail later.

The body age acquisition unit 16 computes the body age in accordance with the procedure described later. The computed activity age and body age are output to the output processing unit 18. The image generation unit 19 of the output processing unit 18 generates image data based on the input activity age and body age. The generated image data is stored in the area E4. The output processing unit 18 displays on the display 20 various items of information such as the generated image data in the area E4 and the like (step S13). This ends the processing.

Next, computation procedures in the above-described units will be described.

### Method of Computing Basal Metabolic Rate

Computation of basal metabolic rate (step S3) performed by the basal metabolic rate acquisition unit 15 will now be described. The basal metabolic rate acquisition unit 15 computes a theoretical basal metabolic rate 531 and a body composition information based basal metabolic rate 532.

Computation of the theoretical basal metabolic rate 531 will also now be described. It is known that a theoretical basal metabolic rate for 1 day can be computed by equation (1), and the basal metabolic rate acquisition unit 15 computes the theoretical basal metabolic rate 531 using equation (1). Note that equation (1) is proposed in Ganpule AA, et al. Interindividual variability in sleeping metabolic rate in Japanese subjects, European Journal of Clinical Nutrition (2007), pp. 1-6.

theoretical basal metabolic rate 531−(0.0481×W+0.0234×H−0.0138×R×0.5473×F+0.1238)×239 (1)

where W denotes weight, H denotes height, R denotes real age, and F denotes gender. Weight W is denoted in kilograms (kg) and height H is denoted in centimeters (cm). F is 1 if the gender is male and is 2 if the gender is female.

Note that the equation for computing the basal metabolic rate is not limited to equation (1), and may be another arithmetic equation. Also, the type and value of the parameters used in the computation equation are not limited to those shown in equation (1), and a configuration may be adopted in which the basal metabolic rate is computed from biological information and the type and value of the parameters are determined empirically from the computed basal metabolic rate, for example, or a configuration may be adopted in which a predetermined value is read out from the coefficient data group 51.

The body composition information based basal metabolic rate 532 is computed from the body composition information of the user measured by the weight/body composition measurement unit 301. As for the method for computing the basal metabolic rate from body composition information, the basal metabolic rate can be computed using fat-free mass measured by the weight/body composition measurement unit 301 in accordance with the equation: basal metabolic rate 532=A×FFM+B (FFM: fat-free mass, A, B: constants).

### Method of Computing Calories Burned

Computation of calories burned by the calories burned computation unit 12 (step S5) will now be described. The calories burned computation unit 12 computes ideal calories burned 521 and measurement calories burned 522.

First, the ideal calories burned 521 is computed using the physical activity level PAL (PAL: Physical Activity Level) for 1 day. It is known that PAL can be computed from total calories burned (unit: kcal) for 1 day/basal metabolic rate (unit: kcal) for 1 day, and that the physical activity level PAL is 1.60 to 1.90 for a “normal” amount of activity. Here, the intermediate value 1.75 is employed as the representative value.

Therefore, if PAL is used, an equation: “ideal calories burned [kcal/day]=ideal basal metabolic rate 531 [kcal/day]×1.75” is satisfied, and the ideal calories burned 521 is derived from this equation.

Next, computation of the measurement calories burned 522 will be described. The measurement calories burned 522 indicates calories burned by the user exercising. The calories burned computation unit 12 computes the measurement calories burned 522 in accordance with the following equation: measurement calories burned (kcal/day)=activity intensity (METs)×weight (kg)×activity duration (hour) for 1 day×1.05 (from Exercise and Physical Activity Guide for Health Promotion “Exercise Guide 2006”, Ministry of Health, Labour and Welfare). Here, activity intensity and activity duration can be acquired from the activity intensity data Mi, and weight can be acquired from physique data 57 in the memory 50.

### Method of Computing Activity Age

Acquisition of activity age (step S7) performed by the activity age acquisition unit 13 will now be described.

The activity age acquisition unit 13 computes activity age in accordance with the following equation. Note that a coefficient k1 of the equation is read out from the coefficient data group 51.

activity age [age]=real age [age]+k1(ideal calories burned 521−measurement calories burned 522)

This equation indicates that “activity age” is a standard (or average) age of persons who, in a predetermined period, burn the total amount of calories burned by the user when active for the same period.

Note that the activity age acquisition unit 13 may acquire the activity age by searching a table, instead of computing activity age through an arithmetic equation. In other words, a table is stored in the memory 50 in advance in which the value of (ideal calories burned 521−measurement calories burned 522) and the activity age computed in accordance with the above-described equation are stored for each real age in association with each other. The activity age acquisition unit 13 may read out the activity age by searching the table based on the real age of the user and the value of (ideal calories burned 521−measurement calories burned 522).

### Method of Computing Body Age

The body age acquisition unit 16 computes the body age in accordance with the following equation. Note that a coefficient k2 of the equation is read out from the coefficient data group 51.

body age [age]=real age [age]+k2(ideal basal metabolic rate 531−body composition information based basal metabolic rate 532)

According to this equation, “body age” represents the age of a body to be computed based on basal metabolic rate. Therefore, the equation indicates that in the case where (ideal basal metabolic rate 531=body composition information based basal metabolic rate 532) is satisfied, the body age denotes the real age and the body matches the user's age. On the other hand, the equation indicates that in the case where (ideal basal metabolic rate 531>body composition information based basal metabolic rate 532) is satisfied, the body composition information based basal metabolic rate 532 is small, and thus the body age exceeds the real age and the body tends to be biologically older than the real age.

Also, the equation indicates that in the case where (ideal basal metabolic rate 531<body composition information based basal metabolic rate 532) is satisfied, the body composition information based basal metabolic rate 531 is large, and thus the body age is less than the real age and the body tends to be biologically younger than the real age.

Generation and Display of Image

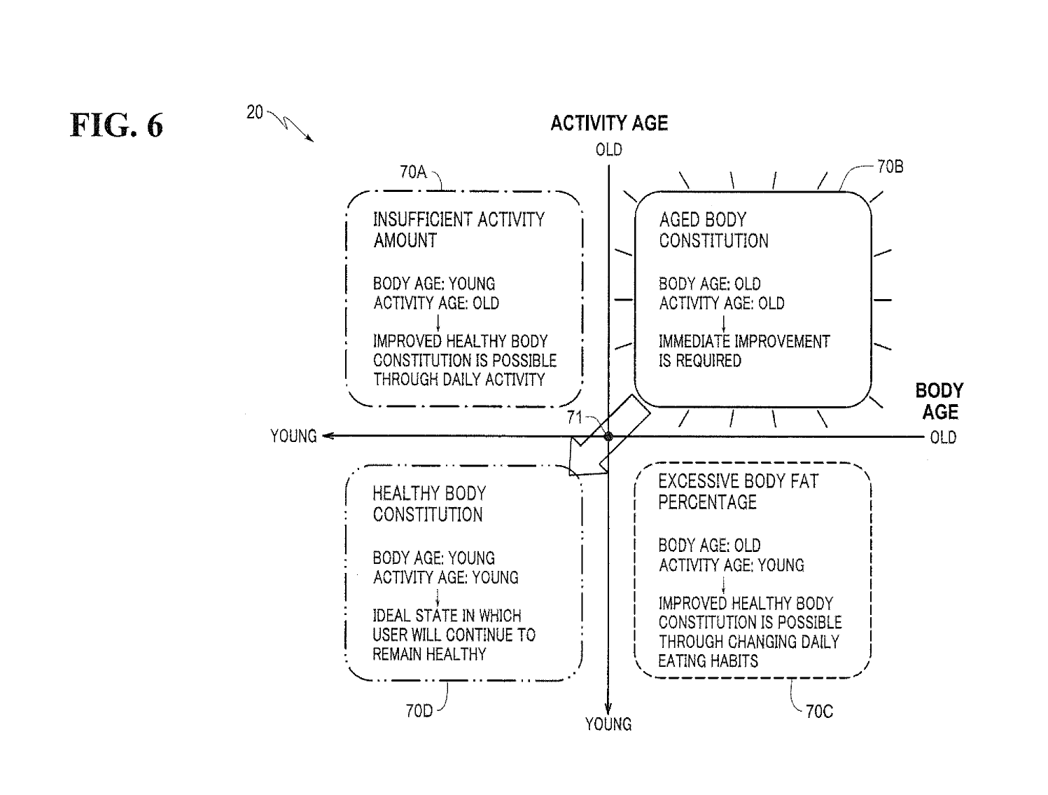

When various ages are acquired in accordance with the above-described procedures, the acquired ages are stored in the area E3. The image generation unit 19 reads out various ages from the area E3, and generates image data for display shown in FIG. 6 using the image data 61 of the area E4.

The image generation unit 19 generates image data including a message of advice relating to meals and activity based on the difference between body age and real age or the difference between activity age and real age, and outputs the generated image data to the output processing unit 18. The output processing unit 18 displays an image on the display 20 based on the image data that has been input. An example of display is shown in FIG. 6.

In FIG. 6, on a two dimensional coordinate plane defined by using an axis representing activity age and an axis representing body age that is orthogonal to the activity age axis, the real age is positioned at a point where these two axes intersect. The two dimensional plane of FIG. 6 is divided into four regions 70A to 70D by the two orthogonal axes. In the each region 70A to 70D, advice information obtained by evaluating activity amount based on a correlation between the body age and the activity age is presented.

The region 70A indicates an insufficient activity amount. Specifically, advice is displayed indicating that the body age is younger than the real age, but the activity age is high (old) and thus the activity amount is insufficient, and that an improved healthy body constitution is possible through daily activity.

The region 70B indicates an aged body constitution. Specifically, advice is displayed indicating that the body age and the activity age are older than the real age (ages are high) and the user has an aged body constitution, and that the activity amount in daily life or body constitution needs to be improved immediately.

The region 70C indicates an excessive body fat percentage. Specifically, advice is displayed indicating that the body age is older (higher) than the real age, but the activity age is young and thus the body fat percentage is excessive, and that an improved healthy body constitution is possible through changing daily eating habits.

The region 70D indicates a healthy body constitution age. Specifically, advice is displayed indicating that the body age and the activity age are younger than the real age, and thus the user is in an ideal state in which he/she will continue to remain healthy in the foreseeable future.

Information on advice displayed in each region may be acquired by searching a table. In other words, a configuration may be adopted in which information on advice is stored in advance in a predetermined table (not shown) in the memory 50 in association with the differences between the body age 59 and the real age 56 and the differences between the activity age 54 and the real age 56, and the image generation unit 19 computes a difference between the body age 59 and the real age 56 or a difference between the activity age 54 and the real age 56 and searches the table based on the computed difference so as to acquire information on the corresponding advice.

In FIG. 6, sections may be clarified by using different colors for displaying each region. Also, the region corresponding to the activity age and the body age of the user (in the case of FIG. 6, region 70B) may be displayed in a different mode from other regions, by being displayed in a blinking manner or the like so as to attract the user's attention.

Also, with FIG. 6, it is possible to arouse the user's attention by displaying an arrow that points toward the region 70D in order to improve his/her life style so that the activity age and the body age point toward the healthy body constitution of the region 70D.

Note that in FIG. 6, the activity age 54 and the body age 59 acquired in steps S7 and S9 may be displayed, or the difference between the body age 59 and the real age 56 and the difference between the activity age 54 and the real age 56 may be displayed.

Modes for displaying advice relating to meals and the amount of activity based on the body age, the activity age and the real age are not limited to FIG. 6.

In other words, if three types of ages, i.e. activity age, body age and real age, are considered from the viewpoint of body composition information, activity age can be considered as an index of the total calories burned (=exercise amount) as shown in the above-described computation equation.

Also, body age can be considered as an index of body fat percentage eating habits). In other words, if the above-described computation equation is used, body age is computed using body composition information (more specifically, fat-free mass), and thus if body fat percentage is high, body age increases, and if body fat percentage is low, body age decreases. Therefore, body age can be considered as body fat percentage. Here, body fat percentage can be improved by changing eating habits to lower fat diet, and therefore, it is possible to present advice about dietary composition by using body age.

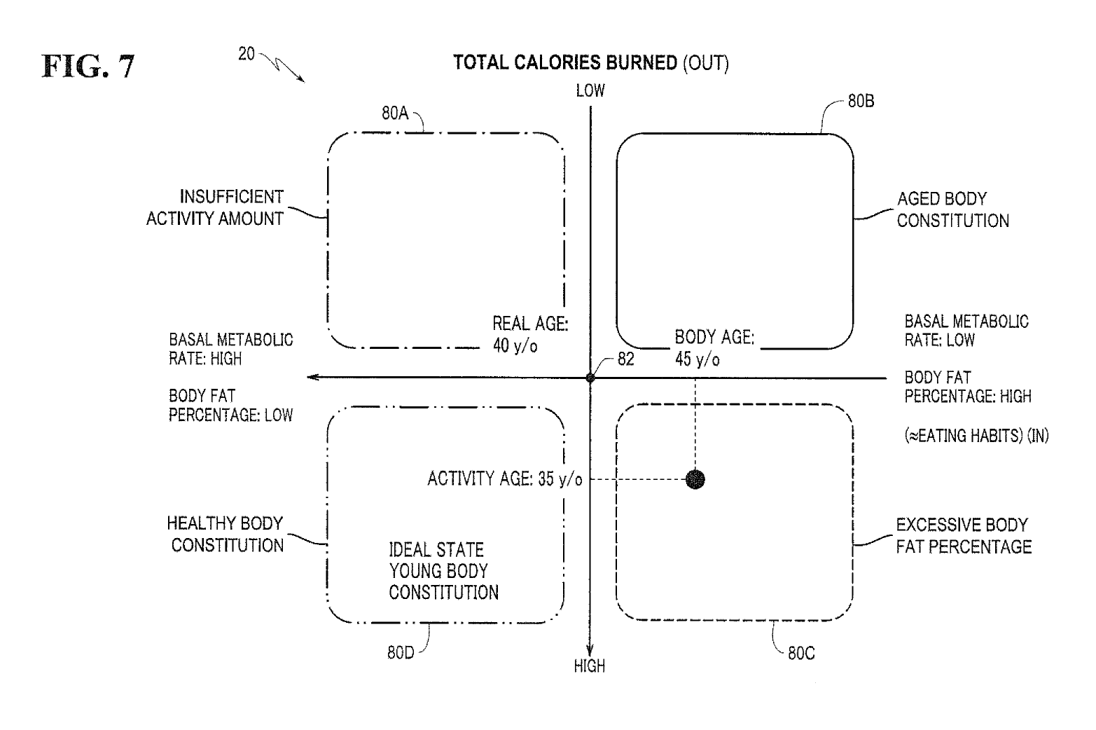

In FIG. 7, an example of display is shown in which the activity age axis and the body age axis of FIG. 6 are respectively replaced by total calories burned and by parameters of basal metabolic rate or body fat percentage, and the regions 70A to 70D are substituted by regions 80A to 80D.

FIG. 7 shows a case in which the acquired activity age and body age are displayed where the real age, body age and activity age are respectively 40 y/o, 45 y/o and 35 y/o, and a mark 81 is plotted and displayed in a region that is closely related to these two ages. It is possible for the user to check whether his/her basal metabolic rate (body fat percentage) is higher or lower than that of the real age by checking the region where the mark 81 is displayed, and to check whether his/her total calories burned is higher or lower than that of real age and thus the user can gain motivation to improve his/her eating habits and the amount of activity.

Also, if the user specifies the displayed mark 81 via the operation unit 30 through a click operation or the like, the output processing unit 18 searches the table described with reference to FIG. 6, reads out the advice corresponding to the region, and displays the readout advice on the display 20. For example, the advice: “There is no problem about the amount of daily activity. Let's aim to have a much younger body by controlling eating habits.” is displayed.

Advice displayed in FIGS. 6 and 7 may be output via audio from the audio output unit 80 together with or instead of a display.

Acquisition of Aging Index

In the present embodiment, the evaluation acquisition unit 17 evaluates the body age of the user, that is, the degree of aging, using the difference between the body age 59 and the real age 56 and the difference between the activity age 54 and the real age 56. Here, the evaluation value computed by the evaluation acquisition unit 17 is referred to as the “aging index”.

The computed aging index is output from the output processing unit 18 via the display 20 or the audio output unit 80. The output processing unit 18 outputs the “aging index” as advice information about improvement of life style indicating that the body age is to improve or worsen in the case of the current life style (the amount of activity, eating habits, and the like) continuing. Computation of aging index and output of advice information on the improvement of life style will be described below.

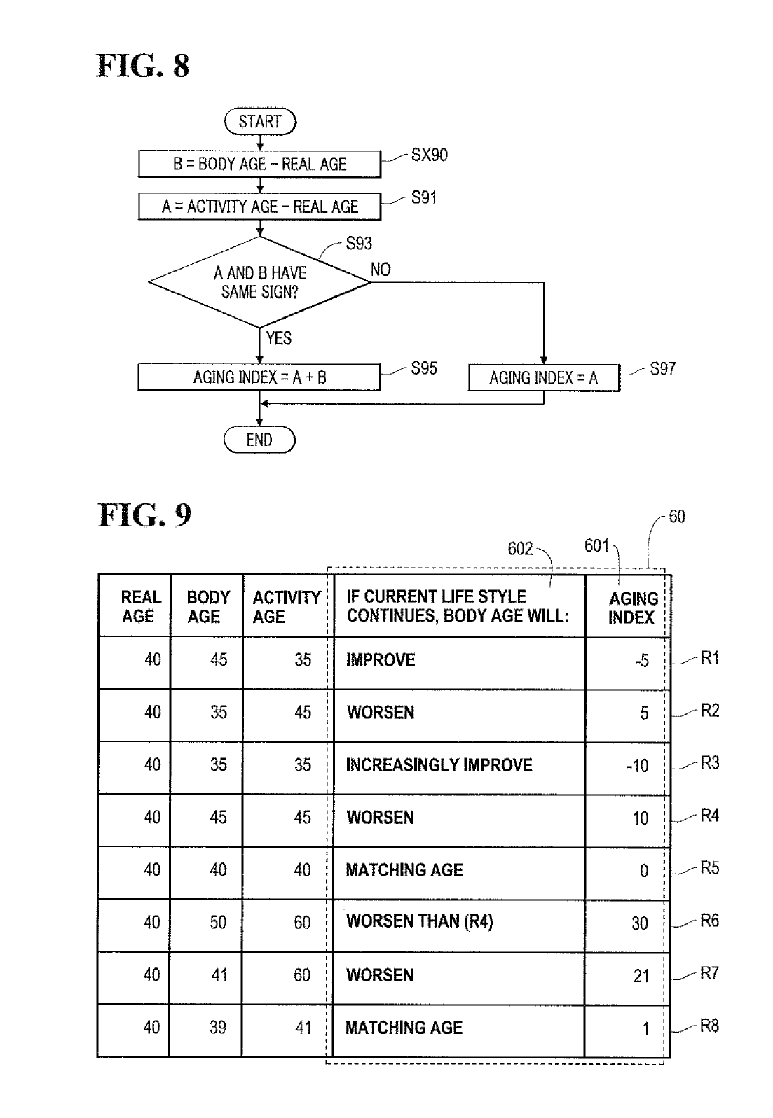

First, the procedure for computing aging index will be described according to the flowchart of FIG. 8. Referring to FIG. 8, the evaluation acquisition unit 17 reads out the body age 59, the real age 56 and the activity age 54 from the memory 50, and computes B=(body age 59−real age 56) (step S90), and computes A=(activity age 54−real age 56) (step S91).

The evaluation acquisition unit 17 then determines whether or not the value A and the value B have the same sign (step S93). If it is determined that the value A and the value B have the same sign (YES in step S93), the evaluation acquisition unit 17 computes aging index=A+B (step S95), and if it is determined that their signs are not the same and they have different signs (NO in step S93), the evaluation acquisition unit 17 computes aging index=A (step S97). This ends the computation of the aging index.

In the present embodiment, when the aging index is computed, advice on the improvement of life style according to the computed aging index is output. In FIG. 9, a portion of the table 60 storing data of advice on the improvement of life style according to aging indices is excerpted and shown as an example.

Referring to FIG. 9, records R1 to R8 having aging index values 601 and advice data 602 corresponding to each aging index value are stored in the table 60. In FIG. 9, for description, the real age 56, the body age 59 and the activity age 54 used for computing aging indices of the records are shown in association with each record, as examples.

The aging indices computed by the evaluation acquisition unit 17 are output to the output processing unit 18. The output processing unit 18 searches the table 60 based on the aging index values to be input and specifies the corresponding record. The output processing unit 18 reads out the advice data 602 to be stored in the specified record, and outputs the readout advice data 602 via the display 20 or the audio output unit 80. Accordingly, it is possible for the user to be made aware of the necessity for an improvement of life style based on activity age and body age.

As shown in FIG. 9, the output processing unit 18 outputs advice indicating that the body age has improved or worsened, using the signs of aging index values. Specifically, advice is output that is divided into +: worsen, 0: matching age, and −: improve.

Modes for displaying aging indices are not limited to this, and other display modes may be adopted. For example, a configuration may be adopted in which each time an aging index is computed, the computed aging index is accumulated and stored together with the measurement date and time, and chronological change in aging indices is displayed using a trend graph, based on the stored information.

Advice based on the aging index of FIG. 9 may be output singularly, or may be output together with the display of FIG. 6 or FIG. 7.

### Another Example of Displaying Acquired Age

Modes for outputting information on activity age or body age are not limited to the output modes of FIGS. 6 and 7. For example, the output processing unit 18 displays on the display 20 the activity age computed by the activity age acquisition unit 13 together with calories burned for 1 day computed by the calories burned computation unit 12, as shown in (A) of FIG. 1. The user is able to judge that the amount of activity (exercise) is appropriate in the case where the displayed activity age indicates the real age or is close to the real age, and is able to judge that the amount of activity (exercise) is insufficient if the displayed activity age greatly exceeds the real age. Accordingly, the user is able to gain the motivation to continue the appropriate amount of activity (exercise).

Although the activity age is displayed in (A) of FIG. 1, the body age acquired by the body age acquisition unit 16 may be displayed instead of or together with the activity age.

Information relating to age that is output is not limited thereto. For example, a value (+5 years old, etc.) obtained by subtracting the activity age (or the body age) from the real age or information indicating the age group (twenties, etc.) to which the activity age (or body age) belongs may be output.

### Other Embodiments

The method for computing and outputting the activity age described using the abovementioned flowchart can also be provided as a program. The program for realizing the method is stored in the memory 50 of the activity meter 100 in advance, and the processing is realized by the CPU 10 reading out the program from the memory 50 and executing the instruction code. This program may be supplied by being downloaded from an external information processing device including the device 200 to the memory 50 via the communication I/F 60 through a communication line.

Also, the device 200 may store such a program and the data shown in. FIG. 4 in the memory 202, the activity age and the body age may be computed in the device 200 by the CPU 201 reading out the program from the memory 202 and executing the instruction code, and the computed activity age and body age may be displayed via the output unit 203 as shown in FIG. 7 or FIG. 8. The data shown in FIG. 4 can be transmitted from the activity meter 100 to the device 200 via the communication I/F 60. Also, the activity age and the body age computed by the device 200 and the output information of FIGS. 7 to 9 may be transmitted to the activity meter 100 and displayed on the display 20 of the activity meter 100.

To allow the device 200 to compute the activity age, the program is provided to the device 200 as a program product recorded on a computer-readable recording medium (not shown) that is attached to the device 200 such as a flexible disk, the CD-ROM 206, a ROM (Read Only Memory) of the memory 202, a RAM (Random Access Memory), or a memory card. Alternatively, the program can also be provided by prerecording the program on a recording medium such as a hard disk (not shown) built into the device 200. Also, the program can also be provided by download to the device 200 from other information processing device via a network.

The embodiments disclosed herein are to be considered in all respects as illustrative and not restrictive. The scope of the invention is defined by the claims rather than by the above description, and all changes that come within the meaning and range of equivalency of the claims are intended to be embraced therein.

### REFERENCE SIGNS LIST

11 activity amount acquisition unit
12 calories burned computation unit
13 activity age acquisition unit
15 basal metabolic rate acquisition unit
16 body age acquisition unit
17 evaluation acquisition unit
18 output processing unit 19 image generation unit
20 display
30 operation unit
31 button
40 acceleration sensor unit
54 activity age
56 real age
59 body age
100 activity meter
301 weight/body composition measurement unit
521 ideal calories burned
522 measured calories burned
531 ideal basal metabolic rate
532 body composition information based basal metabolic rate
Mi activity intensity data

1. An activity meter comprising:
activity amount acquisition means for acquiring an amount of physical activity of a user;
means for acquiring a first index relating to a difference between target calories burned for a unit period and computed calories burned that are computed from the amount of activity acquired by the activity amount acquisition means in the unit period;
means for acquiring a second index relating to a difference between a target value representing a body composition and a measurement value representing a body composition that is measured for the user; and
means for outputting information on an evaluation of the activity amount based on the first index and the second index that have been acquired.

2. The activity meter according to claim 1,
wherein the means for acquiring the first index acquires, as the first index, an activity age representing a standard age of a person who does the same amount of activity as the activity amount acquired in the unit period, using a real age of the user and the difference between the target calories burned and the computed calories burned, and
the means for acquiring the second index acquires, as the second index, a body age indicating a biological age of the user, using the real age of the user and the difference between the target value representing a body composition and the measurement value representing a body composition that is measured for the user.

3. The activity meter according to claim 2, wherein the means for outputting the evaluation information outputs the information on the evaluation of the activity amount based on the real age, the activity age and the body age of the user.

4. The activity meter according to claim 2, wherein the means for outputting the evaluation information outputs the evaluation information in association with a position indicated by the activity age and the body age that have been acquired for the user, on a coordinate plane defined by an axis of the activity age and an axis of body age that is orthogonal to the activity age axis.

5. The activity meter according to claim 4, wherein the means for outputting the evaluation information outputs the evaluation information in association with the position indicated by the activity age and the body age that have been acquired for the user, on a coordinate plane with the activity age axis being substituted by an axis of calories burned and the body age axis being substituted by an axis of the value representing a body composition.

6. The activity meter according to claim 1, wherein the value representing a body composition indicates a basal metabolic rate.

7. The activity meter according to claim 1, wherein the value representing a body composition indicates a body fat percentage.

8. The activity meter according to claim 2, wherein the means for outputting the evaluation information outputs information on an evaluation of an activity amount based on the difference between the activity age and the real age and the difference between the body age and the real age.

9. An activity amount management method for managing an amount of activity of a user with use of a processor, comprising the steps of:
the processor acquiring an amount of physical activity of a user;
the processor acquiring a first index relating to a difference between target calories burned for a unit period and computed calories burned that are computed from the activity amount acquired in the activity amount acquisition step in the unit period;
the processor acquiring a second index relating to a difference between a target value representing a body composition and a measurement value representing a body composition that is measured for the user; and
the processor outputting, on a display, information on an evaluation of the activity amount based on the first index and the second index that have been acquired.

10. A program for causing a processor to execute an activity amount management method, the activity amount management method comprising the steps of:
the processor acquiring an amount of physical activity of a user;
the processor acquiring a first index relating to a difference between target calories burned for a unit period and computed calories burned that are computed from the activity amount acquired in the activity amount acquisition step in the unit period;
the processor acquiring a second index relating to a difference between a target value representing a body composition and a measurement value representing a body composition that is measured for the user; and
the processor outputting on a display information on an evaluation of the activity amount based on the first index and the second index that have been acquired.

11. A system comprising a measurement device that measures an amount of activity of a user and an information processing device,
the measurement device including:
means for measuring an activity intensity of the user; and
means for outputting measurement data in which the activity intensity is associated with a measurement date-time, and
the information processing device including:
means for receiving the measurement data output from the measurement device;
activity amount acquisition means for acquiring an amount of physical activity of a user from the measurement data;
means for acquiring a first index relating to a difference between target calories burned for a unit period and computed calories burned that are computed from the amount of activity acquired by the activity amount acquisition means in the unit period;
means for acquiring a second index relating to a difference between a target value representing a body composition and a measurement value representing a body composition that is measured for the user; and
means for outputting information on an evaluation of the amount of activity based on the first index and the second index that have been acquired.
